(19) United States
(12) Patent Application Publication
(10) Pub. No.: US 2006/0257876 A1
(43) Pub. Date: Nov. 16, 2006

(54) Polymerase-based protocols for generating chimeric and combinatorial...

(75) Inventor: John C. Salerno, Averill Park, NY (US)

Correspondence Address: ELMORE PATENT LAW GROUP, PC, 209 MAIN STREET, N. CHELMSFORD, MA, 01863, US

(73) Assignee: Rensselaer Polytechnis Institute

(21) Appl. No.: 10/544,418

(22) PCT Filed: Feb. 6, 2004
(86) PCT No.: PCT/US04/03502

§ 371 Date: Dec. 9, 2005

Related U.S. Application Data
(60) Provisional application No. 60/445,704, filed on Feb. 6, 2003.
(60) Provisional application No. 60/445,689, filed on Feb. 6, 2003.
(60) Provisional application No. 60/445,703, filed on Feb. 6, 2003.
(60) Provisional application No. 60/446,045, filed on Feb. 6, 2003.
(60) Provisional application No. 60/474,063, filed on May 29, 2003.

(51) Int. Cl. C12Q 1/68; C12P 19/34

(52) U.S. Cl. 435/6; 435/91.2

(57) ABSTRACT

The present invention relates to methods of generating chimeric oligonucleotides without the need for subcloning. The methods of the invention are polymerase-based, and may optionally be adapted for use with reagents available in commercially available mutagenesis kits such as Stratagene's QCM kits, to generate chimeric oligonucleotides in a quick, efficient and cost-effective manner. Major applications of this method include vaccine production, directed evolution and other areas that benefit from the development of diversity.

### RELATED APPLICATIONS

This application claims the benefit of U.S. Provisional Application No. 60/445,704, filed on Feb. 6, 2003, U.S. Provisional Application No. 60/445,689, filed on Feb. 6, 2003, U.S. Provisional Application No. 60/445,703, filed on Feb. 6, 2003, U.S. Provisional Application No. 60/446,045, filed on Feb. 6, 2003, and U.S. Provisional Application No. 60/474,063, filed on May 29, 2003, Docket No. RPI-812, entitled “Parental Suppression via Polymerase-based Protocols for the Introduction of Deletions and Insertions.” The entire teachings of the above applications are incorporated herein by reference.

### BACKGROUND OF THE INVENTION

In recent years a number of methods have come into common use that allow the generation of site directed mutants without subcloning based on polymerase activity. This technology is mature enough to allow the sale of a number of mutagenesis kits that are capable of producing point mutants and in some case insertion and deletion mutants (‘indels’). One such mutagenesis system is supplied by Stratagene (La Jolla, Calif.) and is sold under the name QuikChange® Site Directed Mutagenesis Kit (QCM).

There are currently no comparatively quick, efficient and cost effective procedures for the production of chimeric oligonucleotides. “Chimeric oligonucleotides” are oligonucleotides that contain regions derived from two or more parent genes as opposed to site-directed mutagenized DNA comprising only point mutations or indels. Chimeric polynuceotides are useful in techniques such as “gene shuffling” (see, e.g. Crameri, A., et al., Nature 391 (6664):228-291 (1998)) and other processes aimed at producing proteins with novel properties.

It would be desirable to have quick, efficient and cost effective procedures for producing chimeric oligonucleotides that may optionally take advantage of existing commercially available kits and processes designed for site-directed mutagenesis or other purposes.

### SUMMARY OF THE INVENTION

The present invention relates to methods of generating chimeric oligonucleotides without the need for subcloning and optionally without the need for an in-vitro ligation step. The methods of the invention are polymerase-based, and may optionally be adapted for use with reagents available in commercially available mutagenesis kits such as Stratagene's QCM kits, to generate chimeric oligonucleotides in a quick, efficient and cost-effective manner. In accordance with the invention, the first stage comprises: a) adding a forward primer to a first parent vector comprising a first target gene and carrying out at least one cycle of single-primer linear amplification reaction; and b) separately adding a reverse primer to a second parent vector comprising a second target gene and carrying out at least one cycle of a single-primer linear amplification reaction. The 3′ end of the forward primer is complementary to a region of the first gene of interest in the first parent vector, while the 3′ end of the reverse primer is complementary to a region of the second gene of interest in the second parent vector. One or both of the forward and reverse primers carries a 5′ extension that is complementary to the 5′ end of the other primer, and the priming regions of each of the forward and reverse primers do not significantly overlap, but are contiguous or nearly contiguous with respect to sequence alignment. Both the first target gene and the second target gene are contained within the same cloning site in the same vector of each respective parental DNA strand, and the primer extension reaction for each parental DNA is halted short of the 3′ or 5′ end of the respective gene of interest (depending on the coding strand being copied) by an appropriate blocking oligonucleotide. The blocking oligonucleotides in each primer extension reaction are positioned to allow enough of the respective vector to be copied to produce significant overlap of the linear DNA strands produced in each primer extension reaction. After the production of the respective linear DNA strands in the first and second reactions, the products of each reaction are combined, annealed and in a second stage reaction, extended by one cycle of a primer extension reaction in the presence of blocking oligonucleotides designed to halt extension to preserve sticky ends. The primer extension reaction results in a chimeric oligonucleotide duplex that may be used to transform competent or ultracompetant cells capable of expressing the chimera.

### BRIEF DESCRIPTION OF THE DRAWINGS

The foregoing and other objects, features and advantages of the invention will be apparent from the following more particular description of the preferred embodiments of the invention, as illustrated in the accompanying drawings in which like reference characters refer to the same parts throughout the different views. The drawings are not necessarily to scale, emphasis instead being placed upon illustrating the principles of the invention.

FIG. 1 is a diagram outlining the basic method of the invention characterized by one double ended primer which has a priming sequence and a 5′ extension which is homologous, identical or complementary to the first parental DNA.

FIG. 2 is a diagram outlining an alternative embodiment of the invention characterized by both primers being double ended primers which each have a priming sequence and a 5′ extension which is homologous, identical or complementary to the other parental DNA.

FIG. 3 is a diagram outlining an alternative embodiment of the invention characterized by a second linear amplification reaction of the first amplification products prior to annealing, extension, recircularization and transformation.

FIG. 4 is a diagram outlining an alternative embodiment of the invention characterized by the use of the reaction product of the linear amplification reaction of the first parental DNA as the primer for the linear amplification reaction of the second parental DNA.

FIG. 5 is a diagram outlining an embodiment of the invention characterized by the use of a double ended primer, hybridized to a first parental DNA, subjected to at least one (preferably at least 5, 10 or 20 or more) cycles of linear amplification. The amplification preferably extends into the vector sequences. The product is then subjected to a reverse primer and linear amplification. The amplification product is used as a primer sequence with a second parental DNA, followed by nick repair.

FIGS. 6A, 6B and 6C illustrate methods of the invention wherein multiple primers can be used and combined to facilitate chimeragenesis.

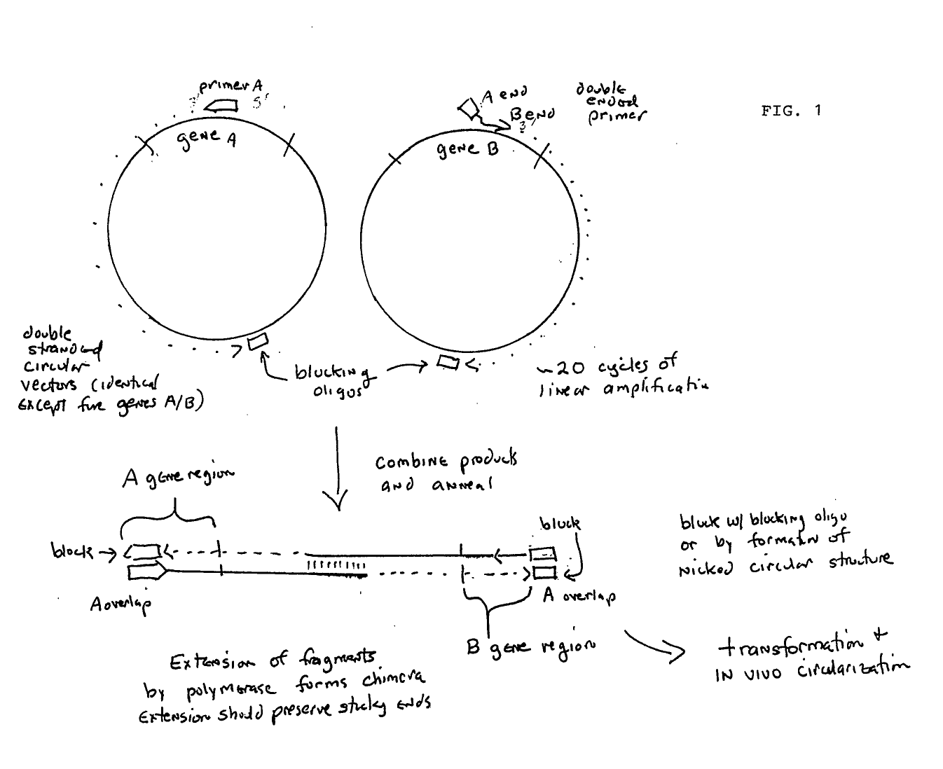

### DETAILED DESCRIPTION OF THE INVENTION

The present invention provides novel methods for the generation of chimeric oligonucleotides without the need for subdoning. In a first aspect, the method of the invention comprises the steps of:
(a) adding a forward, or first, primer and a first blocking oligonucleotide to a first parental DNA comprising a first target sequence, such as a gene, in a first reaction, wherein the forward primer comprises a 3′ region that is complementary to a region of the first target sequence or gene;
(b) adding a reverse, or second, primer and a second blocking oligonucleotide to a second parental DNA comprising a second target sequence, such as a gene, in a second reaction, wherein the reverse primer comprises a 3′ region that is complementary to a region of the second target sequence or gene and wherein one or both of the forward primer and/or the reverse primer comprise a 5′ extension complementary, or substantially homologous in the case of a double primer, to the 5′ region of the other primer;
(c) synthesizing in the first reaction, by means of at least one cycle of a single-primer linear amplification reaction, a first DNA strand comprising at least a portion of the first target sequence or gene and, preferably, at least a portion of a vector sequence;
(d) synthesizing in the second reaction, by means of at least one cycle of a single-primer linear amplification reaction, a second DNA strand comprising at least a portion of the second target sequence or gene and, preferably, at least a portion of a vector sequence, wherein the first and second DNA strands are at least partially complementary, preferably in the vector sequence;
(e) combining the first DNA strand from (c) with the second DNA strand from (d);
(f) annealing the first DNA strand to the second DNA strand to form a partially double-stranded DNA intermediate;
(g) extending by means of at least one cycle of a primer extension reaction, the partially double-stranded DNA intermediate in the presence of third and fourth blocking oligonucleotides that hybridize to the terminal sequences of the first and second DNA strands thereby forming a fully complementary, chimeric DNA duplex comprising the first target gene and the second target gene and further comprising complementary overhanging sticky ends; and
(h) optionally transforming a host cell with the fully complementary, chimeric DNA duplex of step (g).

Alternatively, the first and second DNA strands of step (c) and (d) are subjected to at least one (preferably at least five, more preferably about ten or more) cycles of a primer extension reaction, thereby producing an excess of complements to the first and second DNA strands. These complements are then combined, annealed and extended in steps (e), (f) and (g).

In yet another embodiment, step (b) is omitted, where the reverse primer is added to the second parental DNA. In this embodiment, the product of the linear amplification reaction of step (c) is combined with the second parental DNA. The 5′ and 3′ ends of the amplification product anneal to the second parental DNA, by virtue of sequence complementarity of the primer, such as can be introduced by a 5′ extension sequence, and the vector sequences. The amplification reaction is then continued. The product of this reaction is subjected to extension reaction with a reverse primer, preferably with a 5′ overhang which is complementary to the 5′ end of the amplification product. The use of an optional blocking oligonucleotide that hybridizes to the 5′ end will create sticky ends that facilitate circularization and transformation.

In an alternative to this latter embodiment, the linear amplification product of step (c) is subjected to at least one cycle of linear amplification with a reverse primer. The resulting polynucleotide is then used as a primer sequence with the second parental DNA in an extension reaction, followed by nick repair with a ligase. The resulting DNA strand is then removed from the template, subjected to reverse strand synthesis with, for example, a generic primer that hybridizes to a vector sequence and nick repair. The resulting circular chimeric product can then be transformed.

In one embodiment, the invention further comprises the use of selection enzymes to digest the first and second parental DNAs after the synthesis steps or after the annealing steps.

The terms “linear amplification reaction.” and “single-primer linear amplification reaction” as used herein, refer to a variety of enzyme mediated polynucleotide synthesis reactions that employ pairs of polynucleotide primers to linearly amplify, a given polynucleotide and proceeds through one or more cycles, each cycle resulting in polynucleotide replication. A linear amplification reaction cycle typically comprises the steps of denaturing the double-stranded template, annealing the single primer or primers to the denatured template, and synthesizing polynucleotides from the primers. Thus the term “linear amplification reaction” as used herein is meant to include all of these steps. In the case of a single-primer linear amplification reaction, only one primer is used in each separate single-primer linear amplification reaction. Linear amplification reactions used in the methods of the invention differ significantly from the polymerase chain reaction (PCR). The polymerase chain reaction produces an amplification product that grows exponentially in amount with respect to the number of cycles. Linear cyclic amplification reactions differ from PCR in that the amount of amplification product produced in a linear cyclic amplification reaction is linear with respect to the number of cycles performed. The reaction product accumulation rate laws differ because the products of each cycle in a PCR reaction are templates for the next cycle, while only the parentals are templates in a linear amplification. As in PCR linear amplification reaction cycle typically comprises the steps of denaturing the double-stranded template, annealing the single primer or primers to the denatured template, and synthesizing polynucleotides from the primers. The cycle may be repeated several times so as to produce the desired amount of newly synthesized polynucleotide product. Although linear amplification reactions differ significantly from PCR, guidance in performing the various steps of linear cyclic amplification reactions can be obtained from reviewing literature describing PCR and other polymerase based methods including, PCR: A Practical Approach, M. J. McPherson, et al., IRL Press (1991), PCR Protocols: A Guide to Methods and Applications, by Innis, et al., Academic Press (1990), and PCR Technology: Principals and Applications of DNA Amplification, H. A. Erlich, Stockton Press (1989). PCR is also described in many U.S. patents, including U.S. Pat. Nos. 4,683,195, 4,683,202; 4,800,159; 4,965,188; 4,889,818; 5,075,216; 5,079,352; 5,104,792, 5,023,171; 5,091,310; and 5,066,584, which are hereby incorporated by references. Many variations of amplification techniques are known to the person of skill in the art of molecular biology. These variations include rapid amplification of DNA ends (RACE-PCR), amplification refectory mutation system (ARMS), PLCR (a combination of polymerase chain reaction and ligase chain reaction), ligase chain reaction (LCR), self-sustained sequence replication (SSR), Q-beta amplification, and strand displacement amplification (SDA), and the like. A person of ordinary skill in the art may use these methods to modify the linear amplification reactions used in the methods of the invention.

The term “first target gene” and “second target gene” as used herein refer to two different genes, gene regions or other DNA sequences of interest that are targeted for incorporation into the same chimeric polynucleotide. During the first stage of the first aspect of the invention, each of the first and second target genes is, preferably, independently located within an identical cloning site in an identical vector. The vector comprising the first target gene is referred to herein as “first parental DNA” and the vector comprising the second target gene is referred to herein as the “second parental DNA”. In accordance with the invention, each of the first and second parental DNAs serve as the template in the single-primer linear amplification reaction for producing the first and second DNA strands of the invention. It will be understood by one skilled in the art that each of the first target gene and second target gene could actually comprise more than one gene, however, for convenience only, the invention will be described in terms of two genes.

The terms “forward” and “reverse” primers refer to a pair of oligonucleotide primers that when taken together are useful for generating a desired polynucleotide sequence, such as a chimera comprising two or more target genes, gene regions or other DNA sequence of interest. Generally, the 3′ end of the forward primer comprises a region that is complementary to one target DNA while the reverse primer comprises a region that is complementary to the other target DNA. One or both of the forward and reverse primers comprises a 5′ extension that is complementary to the 5′ region of the other primer or alternatively, to a sequence of one or both target genes. The extensions permit the creation of “sticky ends” which facilitate circularization. In yet other embodiments, double primers can also be used, permitting the use of the amplification product from one DNA strand to prime, amplify or extend a second, or subsequent, DNA strand. The respective priming regions of the forward and reverse primers preferably do not overlap significantly but are contiguous or nearly contiguous with respect to sequence alignment. The primer extension catalyzed by each of the primers during the linear amplification reaction is halted short of the 3′ end of the target gene by an appropriate blocking oligonucleotide which is positioned to allow enough of the vector of each respective target DNA to be copied to produce significant overlap of the resulting DNA strands.

In accordance with the invention, the forward primer and a first blocking oligonucleotide are added to the first parental DNA in a first reaction and the reverse primer and a second blocking oligonucleotide are added to the second parental DNA in a second reaction that is separate from the first reaction. At least one cycle, preferably at least ten cycles, and more preferably at least 20 cycles, of single-primer linear amplification (including denaturing, primer extension and annealing as discussed above) is carried out in each of the first and second reactions. As discussed above, the first and second parental DNA differ only by the target gene located in the respective cloning site of each parental DNA (i.e. the noncloning vector regions and cloning sites of each parental DNA are identical, but the specific gene located within the cloning site is different). The first blocking oligonucleotide in the first reaction is complementary to any region downstream from the forward primer so long as the blocking oligonucleotide stops extension of the forward primer prior to 5′ end of the first target gene and the second blocking oligonucleotide in the second reaction is complementary to the region of the target DNA prior to the 3′ end of the second target gene. This assumes that the “forward primer in the first reaction is homologous to the gene sequence of interest and complementary to the reverse strand of the gene of interest, and that the primer in the second reaction as the opposite direction (i.e. is complementary to the coding strand). One skilled in the art will recognize that the either of the forward and reverse primers can be designed to be complementary to either the coding strand or the reverse strand and that the designation of forward and reverse primers in an association with a particular first or second reaction is for ease of discussion. The blocking oligonucleotides are preferably positioned to allow enough of each respective vector to be copied in order to result in significant overlap of the resulting first and second DNA strands. Thus, when the reaction product of the first reaction (defined herein as the “first DNA strand” comprising the first target gene) is combined and annealed with the reaction product of the second reaction (defined herein as the “second DNA strand” comprising the second target gene) a partially double-stranded DNA intermediate is formed. This DNA intermediate is partially double-stranded because only the overlapping region of each respective DNA strand is double stranded.

A second stage primer extension reaction (preferably only one cycle) is then carried out to extend each of the first and second DNA strands of the partially double stranded DNA intermediate thereby forming a fully complementary chimeric DNA duplex comprising the first and second target genes. Preferably, third and fourth blocking oligonucleotides can be added to the primer extension reaction to block the primer extension to for the purpose of preserving overhanging sticky ends of the chimeric DNA duplex that are useful for is in vivo recircularization and nick repair upon transformation of the chimeric DNA duplex into a host cell. These blocking oligonucleotides should be selected to insure that the resulting circularized polynucleotide creates the desired sequence. It is not necessary to perform a ligation reaction prior to the transformation the host cells.

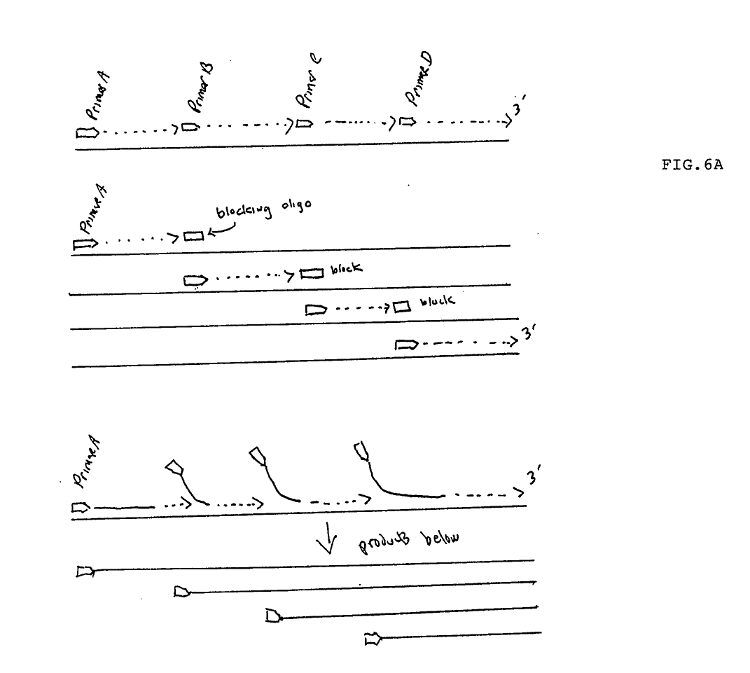

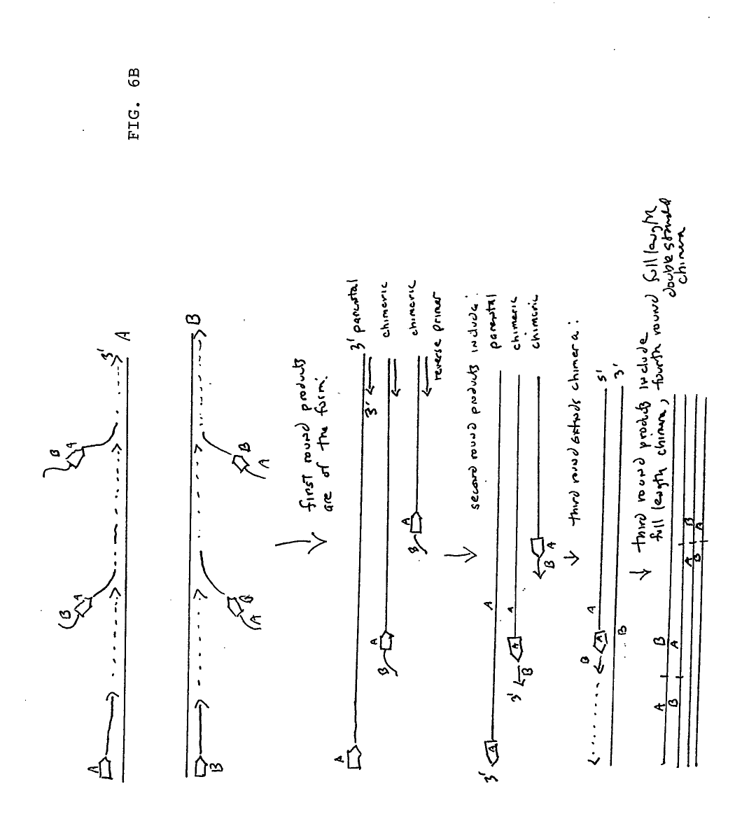

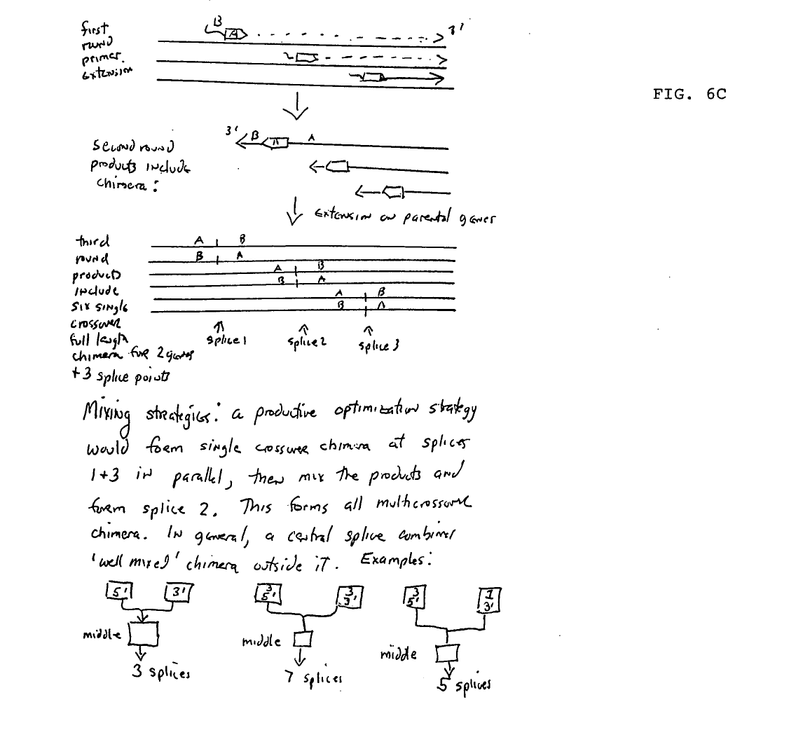

The process can be easily adapted to use multiple primers, providing multiple cross overs between polynucleotides, as illustrated in FIGS. 6A, 6B and 6C. Accordingly, one, two, three, four or more primers (such as the double ended, or 5′ extension, primers described herein) can be used to extend a parental DNA, optionally in the presence of a blocking oligonucleotide, to control the extension reaction. These products can then be used, for example, to extend a second or subsequent parental DNA strand, thereby creating a chimeric sequence, as depicted in FIG. 6A. Use of strand displacing conditions or non-strand displacement conditions controls the length of the resulting product and can be used to manipulate the placement of the cross-overs obtained.

The host cells may be prokaryotic or eukaryotic. Preferably the host cells are prokaryotic, and preferably, the host cells for transformation are E. coli cells. In preferred embodiments, the cells are competent or ultracompetant cells. Ultracompetant cells such as the SL10-Gold® ultracompetetant cells available from Stratagene (La Jolla, Calif.) are particularly useful for the transformation of large DNA molecules with high efficiency. Techniques for preparing and transforming competent single cell microorganisms are well know to the person of ordinary skill in the art and can be found, for example, in Sambrook et al., Molecular Cloning: A Laboratory Manual Coldspring Harbor Press, Coldspring Harbor, N.Y. (1989), Harwood Protocols For Gene Analysis, Methods In Molecular Biology Vol. 31, Humana Press, Totowa, N.J. (1994), and the like. Frozen competent cells may be transformed so as to make the methods of the invention particularly convenient.

The term “oligonucleotide” as used herein with respect to the mutagenic primer the complementary primer and the blocking oligonucleotides is used broadly. Oligonucleotides include not only DNA but various analogs thereof. Such analogs may be base analogs and/or backbone analogs, e.g., phosphorothioates, phosphonates, and the like. Techniques for the synthesis of oligonucleotides, e.g., through phosphoramidite chemistry, are well known to the person ordinary skilled in the art and are described, among other places, in Oligonucleotides and Analogues: A Practical Approach, ed. Eckstein, IRL Press, Oxford (1992). Preferably, the oligonucleotide used in the methods of the invention are DNA molecules.

The terms “first”, “second”, “third” and “fourth” blocking oligonucleotides as used herein refer to appropriate oligonucleotides that are designed to halt primer extension at a desired point in each of the reactions as is described herein. It is understood that the first, second, third and fourth blocking oligonucleotides may each be the same or different, and that terms “first”, “second” “third” and “fourth” as used herein are used merely for convenience to indicate that the blocking oligonucleotide used in each reaction of the invention has the function of halting primer extension at a desired location in that particular reaction.

In accordance with the invention, parental DNA strands used as templates during linear amplification reactions may optionally be digested during the method of the invention. By performing the digestion step, the number of transformants containing non-mutagenized polynucleotides is generally reduced. The term “digestion” as used herein in reference to the enzymatic activity of a selection enzyme is used broadly to refer both to (i) enzymes that catalyze the conversion of a polynucleotide into polynucleotide precursor molecules and to (ii) enzymes capable of catalyzing the hydrolysis of at least one bond on polynucleotides so as to interfere adversely with the ability of a polynucleotide to replicate (autonomously or otherwise) or to interfere adversely with the ability of a polynucleotide to be transformed into a host cell. Restriction endonucleases are an example of an enzyme that can “digest” a polynucleotide. Typically, a restriction endonuclease that functions as a selection enzyme in a given situation will introduce a specific single cleavage into the phosphodiester backbone of the template strands that are digested.

The term “selection enzyme” refers to an enzyme capable of catalyzing the digestion of a polynucleotide template for mutagenesis, but not significantly digesting newly synthesized mutagenized polynucleotide strands. Examples of selection enzymes include restriction endonucleases. One suitable selection enzyme for use in the parental strand digestion step is the restriction endonuclease Dpn I, which cleaves the polynucleotide sequence GATC only when the adenine is methylated (6-methyl adenine). Suitable selection enzymes are provided with commercially available mutagenesis kits such as the QuikChange® Site Directed Mutageneisis System kit supplied by Stratagene (La Jolla, Calif.). A variety of suitable selection enzymes may be purchased from a myriad of companies as is known in the art.

In another embodiment, an alternate selection step may be used. In this embodiment, each parental DNA comprises one “live” and one “dead” restriction site (assuming the respective target gene did not contain the same restriction sites) or alternatively one “live” and “one” dead antibiotic resistance site. The first and second blocking oligonucleotides are designed such that each respective blocking oligonucleotide is located between the selection sites (either resistance or restriction). Only the chimera produced in accordance with the method of the invention will have either both antibiotic sites or alternatively, neither of the restriction sites.

The primers of the invention are preferably about 20-50 bases in length, and more preferably about 25 to 45 bases in length. However, in certain embodiments of the invention, it may be necessary to use primers that are less than 20 bases or greater than 50 bases in length so as to obtain the mutagenesis result desired. The primers may be of the same or different lengths. In some embodiments, the primers are preferably 5′ phosphorylated. 5′ phosphorylation may be achieved by a number of methods well known to a person of ordinary skill in the art, e.g., T-4 polynucleotide kinase treatment. Preferably, the mutagenizing region of the primers are flanked by about 10-15 bases of correct, i.e., non-mismatched, sequence so as to provide for the annealing of the primer to the template DNA strands for mutagenesis. In preferred embodiments of subject methods, the GC content of primers is at least 40%, so as to increase the stability of the annealed primers. Preferably, the primers are selected so as to terminate in one or more G or C bases. Very high GC content (over 70%), or runs of more than five successive GC bases, are not desirable since this decreases specificity.

In one embodiment of the invention, the invention may be carried out using the reagents provided in commercially available mutagenesis kits such as Stratagene's QuikChange® II XL Site Directed Mutagenesis Kit. However, the present invention may be practiced without the use of a commercially available kit so long as a high fidelity polymerase and high competence cells are used in the present method.

The single-primer linear cyclic amplification reaction may be catalyzed by a thermostable or non-thermostable high-fidelity polymerase. Preferably, the polymerase used is a thermostable polymerase. The polymerase used may be isolated from naturally occurring cells or may be produced by recombinant DNA technology. The use of Pfu DNA polymerase (Stratagene, La Jolla, Calif.), a DNA polymerase naturally produced by the thermophilic archae Pyrococcus furiosus, is particularly preferred for use in the linear cyclic amplification reaction steps of the claimed invention. Other high fidelity polymerases suitable for use in the present invention include, but are not limited to, KOD HiFi (EMD Biociences Inc, Madison, Wis.), MA), Phusion™ High-Fidelity Polymerase (Finnzymes, Espoo, Finland).

The single-primer linear amplification reactions as employed in the methods of the invention are preferably carried out to the limits imposed by the polymerase properties and the amplification conditions. Preferably the number of cycles in the linear cyclic amplification reaction step is at least 10 cycles, more preferably at least 20 cycles, and even more preferably at least 25 or more cycles. The limitation on the optimum cycle number is specific to the application and is imposed by runaway PCR artifact triggered by low probability nonspecific binding. This is a problem primarily in very GC rich regions, and can be overcome by decreasing the number of cycles and increasing the parental DNA template present in the reaction.

The second stage primer extension reaction of the invention is preferably carried out for only one cycle. Appropriate reaction conditions are maintained throughout the various stages of the invention to maximize desirable reaction products produced at each stage while minimizing the production of artifact.

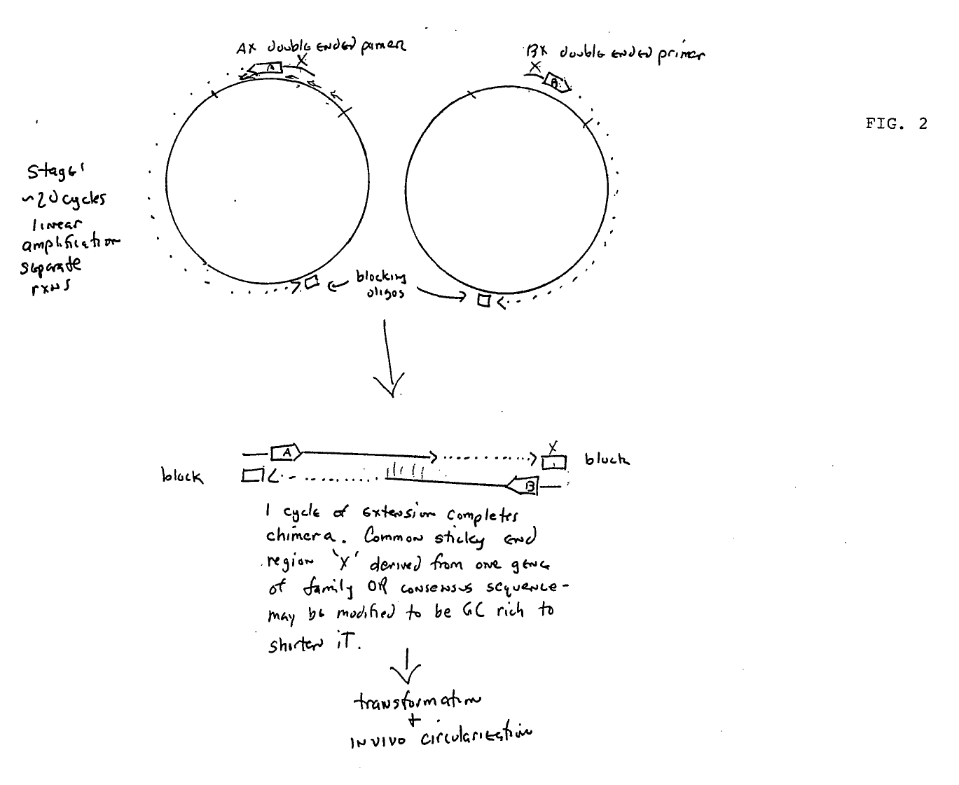

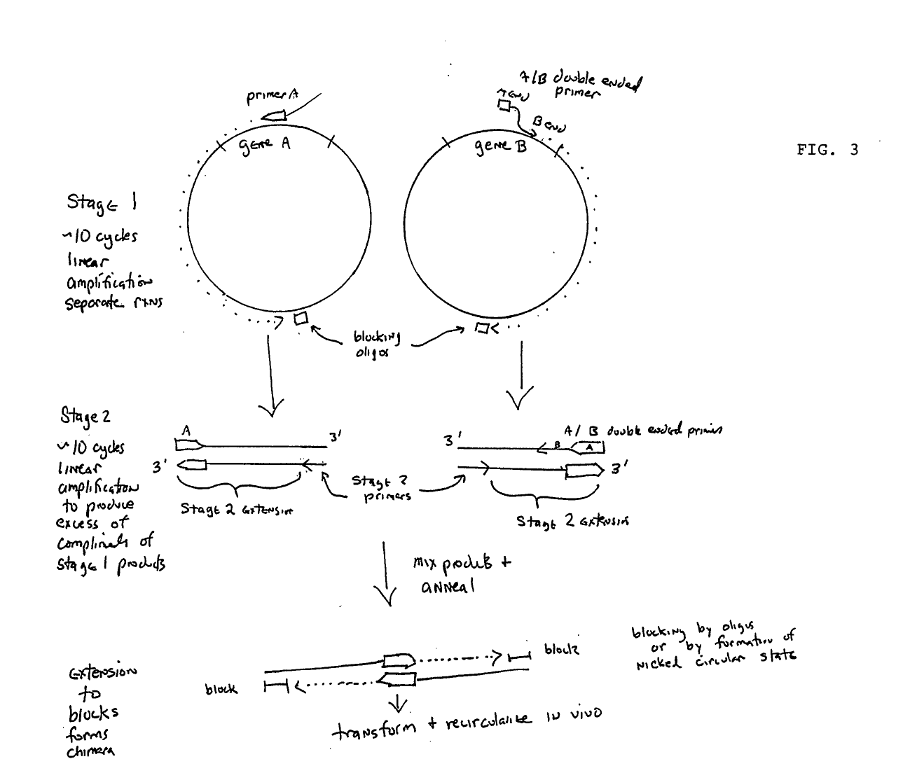

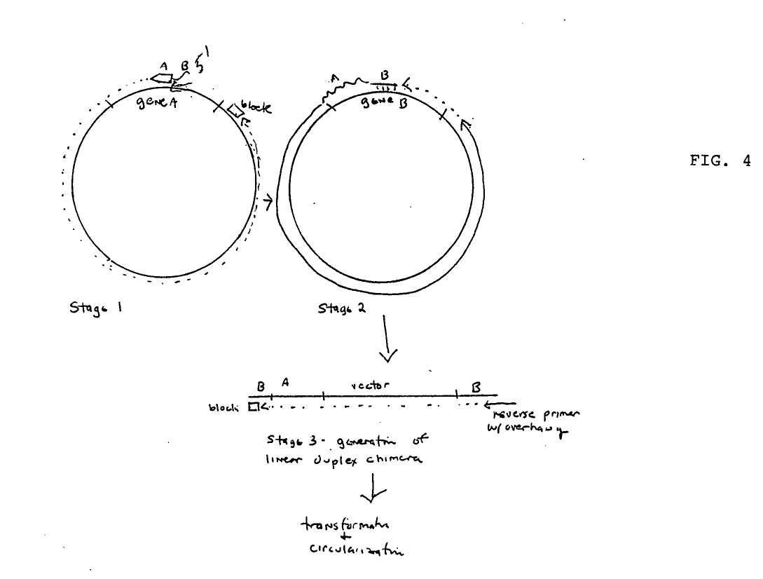

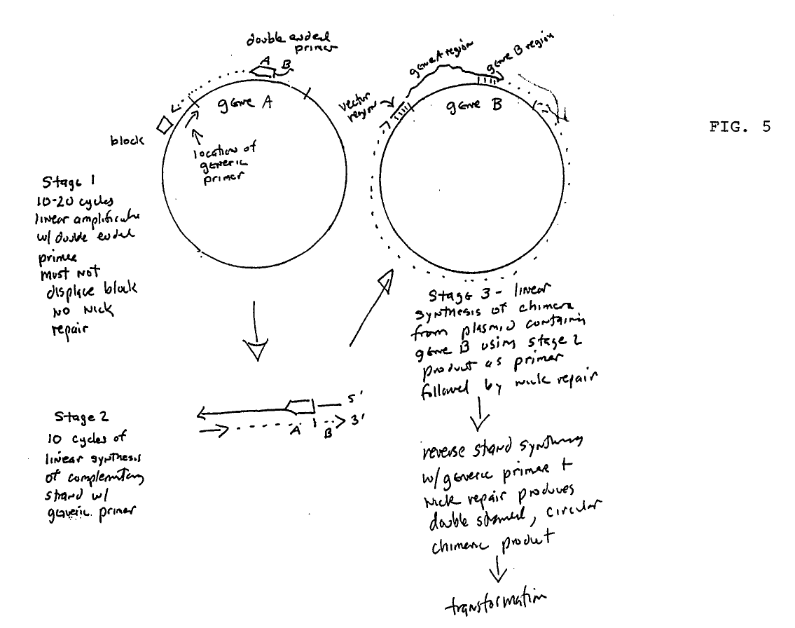

In a second aspect of the invention as shown in FIG. 2, chimeric DNA is generated in three stages. The advantage of this embodiment is that it requires fewer oligonucleotides.

In the first stage of this second aspect of the invention, a forward primer and a first blocking oligonucleotide are added to a first parental DNA comprising a vector region, a cloning site and a first target gene within the cloning site. The 3′ end of the forward primer used in the first stage is complementary to the first target gene. The forward primer further comprises a 5′ tail region that is complementary to all or a portion of a second target gene. The first blocking oligonucleotide is designed to halt primer extension just prior to the 3′ end of the first target gene. At least one cycle of a single-primer linear amplification reaction is used to synthesize a DNA strand comprising the forward primer and extending to the first blocking oligonucleotide.

In the second stage, a second parental DNA comprising a second target gene but having an a vector region and cloning site that are identical to the first parental DNA is used as a template to extend the DNA strand produced in the first stage to copy the remaining vector and the second target gene thereby forming a single-stranded DNA comprising the first and second target genes.

In the third stage, a reverse primer comprising a 5′ overhang is used in conjunction with a blocking oligonucleotide to copy the single-stranded DNA produced in the second stage thereby forming a chimeric DNA duplex with sticky ends that may be used to transform competent or ultracompetent host cells for further processing.

Another aspect of the invention is to provide kits for performing the combinatorial mutagenesis methods of the invention. The kits of the invention provide one or more of the enzymes or other reagents for use in performing the subject methods. The kits may contain reagents in pre-measured amounts so as to ensure both precision and accuracy when performing the subject methods. Kits may also contain instructions for performing the methods of the invention. In one embodiment, kits of the invention comprise at least one polymerase and instructions for carrying out the method. The kits may also comprise a DNA vector comprising a cloning site, ultracompetent cells and blocking oligonucleotides complementary to regions of the DNA vector. Kits of the invention may also comprise individual nucleotide triphosphates, mixtures of nucleoside triphosphates (including equimolar mixtures of dATP, dTTP, dCTP and dGTP), and concentrated reaction buffers. In a preferred embodiment, the kits comprise at least one DNA polymerase, concentrated reaction buffer, a nucleoside triphosphate mix of the four primary nucleoside triphosphates in equal amounts, frozen competent or ultracompetent cells and instructions for carrying out the method.

One skilled in the art will appreciate the many advantages that the method of the invention provides. For example, the improved site-directed mutagenesis methods of the invention are useful in protein and enzyme engineering technologies for the production of industrial proteins and enzymes such as detergent enzymes, enzymes useful for neutralizing contaminants and enzymes useful as fuel additives. Likewise, the methods of the invention are useful in protein engineering technologies for the production of proteins and enzymes useful and the food and life sciences industries such as primary and secondary metobolites useful in the production of antibiotics, proteins and enzymes for the food industry (bread, beer) and combinatorial arrays of proteins for useful in generating multiple epitopes for vaccine production.

The methods of the invention can also be used in the production of mutagenized fusion proteins. A DNA sequence targeted for mutagenesis is tagged or fused with the DNA sequence encoding a known protein (e.g. maltose binding protein (MBP) or green fluorescent protein (GFP)). For example, vectors with a GFP gene adjacent to a cloning site would allow easy conversion of a vector for expression of a target gene to one of several possible target gene-GFP mutants with different linkers. These in turn could be targeted to different cell locations by modifications of the opposite (usually N) terminus. Kits designed identify fusion proteins are advantageously used to identify and isolate the mutagenized protein of interest in vitro (western blots) or in tissue using anti-GFP.

The patent and scientific literature referred to herein establishes the knowledge that is available to those with skill in the art. All United States patents and published or unpublished United States patent applications cited herein are hereby incorporated by reference. All published foreign patents and patent applications cited herein are hereby incorporated by reference. All other published references, documents, manuscripts and scientific literature cited herein are hereby incorporated by reference.

While this invention has been particularly shown and described with references to preferred embodiments thereof, it will be understood by those skilled in the art that various changes in form and details may be made therein without departing from the scope of the invention encompassed by the appended claims.

What is claimed is:

1. A method of generating chimeric oligonucleotides comprising the steps of:
(a) adding a forward primer and a first blocking oligonucleotide to a first parental DNA comprising a first target gene in a first reaction, wherein the forward primer comprises a 3′ region that is complementary to a region of the first target gene and wherein the first blocking oligonucleotide halts the primer extension prior to the 3′ end of the first target gene;
(b) adding a reverse primer and a second blocking oligonucleotide to a second parental DNA comprising a second target gene in a second reaction, wherein the reverse primer comprises a 3′ region that is complementary to a region of the second target gene, wherein the blocking oligonucleotide halts primer extension just prior to the 3′ end of the second target gene, wherein one or both of the forward primer and the reverse primer comprise a 5′ extension complementary to the 5′ region of the other primer and wherein the second parental DNA is identical to the first parental DNA except for the target gene;
(c) synthesizing in the first reaction, by means of at least one cycle of a single-primer linear amplification reaction, a first DNA strand comprising the first target gene;
(d) synthesizing in the second reaction, by means of at least one cycle of a single-primer linear amplification reaction, a second DNA strand comprising the second target gene, wherein the first and second DNA strands are at least partially complementary and wherein one or both of the first and second DNA strands comprise a region of overlap with the other gene;
(e) combining the first DNA strand from (c) with the second DNA strand from (d);
(f) annealing the first DNA strand to the second DNA strand to form a partially double-stranded DNA intermediate; and
(g) extending by means of at least one cycle of a primer extension reaction, the partially double-stranded DNA intermediate in the presence of third and fourth blocking oligonucleotides designed to preserve sticky ends, thereby forming a fully complementary, chimeric DNA duplex comprising the first target gene and the second target gene and further comprising overhanging sticky ends.

2. The method of claim 1 further comprising transforming an ultracompetent host cell with the chimeric DNA duplex of step (g).

3. The method of claim 1 wherein the single primer linear amplification reaction of steps (c) and (d) are each repeated for at least 2-0 cycles.

4. The method of claim 1 wherein the primer extension reaction of step (g) is carried out for 1 cycle.

5. The method of claim 1 wherein the linear amplification reactions of steps (c) and (d) are catalyzed by pfu DNA polymerase.

6. The method of claim 1 wherein the primer extension reaction of step (g) is catalyzed by pfu DNA polymerase.

7. A kit for use in the method of claim 1 comprising, a DNA polymerase, and instructions for carrying out the method.

8. The kit of claim 7 further comprising competent cells.

9. The kit of claim 7 further comprising a DNA vector comprising a cloning site and blocking oligonucleotides complementary to a region of the DNA vector.

10. The kit of claim 7 further comprising concentrated buffers.

11. The kit of claim 7 further comprising individual nucleotide triphosphates, or mixtures of nucleoside triphosphates.

12. A method of using a kit comprising a DNA polymerase and instructions for carrying out the method, in a method comprising the steps of:
(a) adding a forward primer and a first blocking oligonucleotide to a first parental vector comprising a first target gene in a first reaction, wherein the forward primer comprises a 3′ region that is complementary to a region of the first target gene and wherein the blocking oligonucleotide halts the primer extension just prior to the 3′ end of the first target gene;
(b) adding a reverse primer and a second blocking oligonucleotide to a second parental vector comprising a second target gene in a second reaction, wherein the reverse primer comprises a 3, region that is complementary to a region of the second target gene, wherein the blocking oligonucleotide halts primer extension just prior to the 3′ end of the second target gene and wherein one or both of the forward primer and the reverse primer comprise a 5′ extension complementary to the 5′ region of the other primer;
(c) synthesizing in the first reaction, by means of at least one cycle of a single-primer linear amplification reaction, a first DNA strand comprising the first target gene;
(d) synthesizing in the second reaction, by means of at least one cycle of a single-primer linear amplification reaction, a second DNA strand comprising the second target gene, wherein the first and second DNA strands are at least partially complementary and wherein one or both of the first and second DNA strands comprise the first target gene and the second target gene;
(e) combining the first DNA strand from (c) with the second DNA strand from (d);
(f) annealing the first DNA strand to the second DNA strand to form a partially double-stranded DNA intermediate; and
(g) extending by means of at least one cycle of a primer extension reaction, the partially double-stranded DNA intermediate in the presence of third and fourth blocking oligonucleotides designed to preserve sticky ends, thereby forming a fully complementary, chimeric DNA duplex comprising the first target gene and the second target gene and further comprising overhanging sticky ends.

13. The method of claim 9 wherein the kit further comprises competent cells.

14. The method of claim 10 further comprising the step of transforming a host cell with the chimeric DNA duplex of step (g).

15. A method of generating chimeric oligonucleotides comprising the steps of:
(a) adding a forward primer and a first blocking oligonucleotide to a first parental DNA comprising a vector region, a cloning site and a first target gene within the cloning site in a first reaction, wherein the forward primer comprises a 3′ region that is complementary to a region of the first target gene and a 5′ tail region that is complementary to all or a portion of the second target gene, and wherein the blocking oligonucleotide halts the primer extension just prior to the 3′ end of the first target gene.
(b) synthesizing in the first reaction, by means of at least one cycle of a single-primer linear amplification reaction, a DNA strand comprising the forward primer and the region of the first parental DNA up to the blocking oligonucleotide;
(c) combining the DNA strand with a second parental DNA comprising a second target gene in a separate second reaction wherein the second parental DNA comprises a vector region and a cloning site that is identical to the first parental DNA;
(d) extending in the second reaction, by means of at least one cycle of single primer linear amplification reaction, the DNA strand to copy all or a portion of the second target gene thereby forming a single-stranded DNA comprising the first and second target genes;
(e) reacting by means of a single cycle of a primer extension reaction, the single-stranded DNA with a reverse primer comprising a 5′ overhang;
(f) extending the reverse primer to a second blocking oligonucleotide thereby forming a chimeric DNA duplex with sticky ends.

16. The method of claim 15, further comprising transforming an ultracompetent host cell with the chimeric DNA duplex of step (f).

17. The method of claim 15 wherein the single primer linear amplification reaction of steps (b) and (d) are each repeated for at least 20 cycles.

18. A method for the generation of chimeric oligonucleotides comprising the steps of:
(a) adding a forward primer and a first blocking oligonucleotide to a first parental DNA comprising a first target sequence or gene in a first reaction, wherein the forward primer comprises a 3′ region that is complementary to a region of the first target sequence or gene;
(b) adding a reverse primer and a second blocking oligonucleotide to a second parental DNA comprising a second target sequence or gene in a second reaction, wherein the reverse primer comprises a 3′ region that is complementary to a region of the second target sequence or gene and wherein one or both of the forward primer and/or the reverse primer comprise a 5′ extension complementary, or substantially homologous in the case of a double primer, to the 5′ region of the other primer;
(c) synthesizing in the first reaction, by means of at least one cycle of a single-primer linear amplification reaction, a first DNA strand comprising at least a portion of the first target sequence or gene and, optionally, at least a portion of a vector sequence;
(d) synthesizing in the second reaction, by means of at least one cycle of a single-primer linear amplification reaction, a second DNA strand comprising at least a portion of the second target sequence or gene and, optionally, at least a portion of a vector sequence, wherein the first and second DNA strands are at least partially complementary, optionally in the vector sequence;
(e) combining the first DNA strand from (c) with the second DNA strand from (d);
(f) annealing the first DNA strand to the second DNA strand to form a partially double-stranded DNA intermediate;
(g) extending by means of at least one cycle of a primer extension reaction, the partially double-stranded DNA intermediate in the presence of third and fourth blocking oligonucleotides that hybridize to the terminal sequences of the first and second DNA strands thereby forming a fully complementary, chimeric DNA duplex comprising the first target gene and the second target gene and further comprising complementary overhanging sticky ends; and
(h) optionally transforming a host cell with the fully complementary, chimeric DNA duplex of step (g).

19. A method for the generation of chimeric oligonucleotides comprising the steps of:
(a) adding a forward primer and a first blocking oligonucleotide to a first parental DNA comprising a first target sequence or gene in a first reaction, wherein the forward primer comprises a 3′ region that is complementary to a region of the first target sequence or gene;
(b) adding a reverse primer and a second blocking oligonucleotide to a second parental DNA comprising a second target sequence or gene in a second reaction, wherein the reverse primer comprises a 3′ region that is complementary to a region of the second target sequence or gene and wherein one or both of the forward primer and/or the reverse primer comprise a 5′ extension complementary, or substantially homologous in the case of a double primer, to the 5′ region of the other primer;
(c) synthesizing in the first reaction, by means of at least one cycle of a single-primer linear amplification reaction, a first DNA strand comprising at least a portion of the first target sequence or gene and, optionally, at least a portion of a vector sequence and subjecting the product to at least one cycle of a linear amplification reaction;
(d) synthesizing in the second reaction, by means of at least one cycle of a single-primer linear amplification reaction, a second DNA strand comprising at least a portion of the second target sequence or gene and, optionally, at least a portion of a vector sequence, wherein the first and second DNA strands are at least partially complementary, optionally in the vector sequence, and subjecting the product to at least one cycle of a linear amplification reaction;
(e) combining the products of steps (c) and (d);
(f) annealing the first DNA strand to the second DNA strand to form a partially double-stranded DNA intermediate;
(g) extending by means of at least one cycle of a primer extension reaction, the partially double-stranded DNA intermediate in the presence of third and fourth blocking oligonucleotides that hybridize to the terminal sequences of the first and second DNA strands thereby forming a fully complementary, chimeric DNA duplex comprising the first target gene and the second target gene and further comprising complementary overhanging sticky ends; and
(h) optionally transforming a host cell with the fully complementary, chimeric DNA duplex of step (g).

20. A method for the generation of chimeric oligonucleotides comprising the steps of:
(a) adding a forward primer and a first blocking oligonucleotide to a first parental DNA comprising a first target sequence or gene in a first reaction, w herein the forward primer comprises a 3′ region that is complementary to a region of the first target sequence or gene;
(b) synthesizing in the first reaction, by means of at least one cycle of a single-primer linear amplification reaction, a first DNA strand comprising at least a portion of the first target sequence or gene and, optionally, at least a portion of a vector sequence and subjecting the product to at least one cycle of a linear amplification reaction;
(c) adding the product of step (b) to a second parental DNA comprising a second target sequence or gene in a second reaction and subjecting the product to an extension reaction, thereby forming a chimeric polynucleotide;
(d) adding a reverse primer to the chimeric polynucleotide produced by step (c) and subjecting the product to an extension reaction, optionally in the presence of a blocking oligonucleotide; and
(e) optionally circularizing and transforming a host cell with the fully complementary, chimeric DNA duplex of step (d).

21. A method for the generation of chimeric oligonucleotides comprising the steps of:
(a) adding a forward primer and a first blocking oligonucleotide to a first parental DNA comprising a first target sequence or gene in a first reaction, wherein the forward primer comprises a 3′ region that is complementary to a region of the first target sequence or gene and a 5′ extension sequence which is complementary to the 5′ region of a second parental DNA sequence;
(b) synthesizing in the first reaction, by means of at least one cycle of a single-primer linear amplification reaction, a first DNA strand comprising at least a portion of the first target sequence or gene and, optionally, at least a portion of a vector sequence and subjecting the product to at least one cycle of a linear amplification reaction with a reverse primer;
(c) adding the product of step (b) to a second parental DNA comprising a second target sequence or gene in a second reaction and subjecting the product to an extension reaction, thereby forming a chimeric polynucleotide;
(d) adding a reverse primer to the chimeric polynucleotide produced by step (c) and subjecting the product to an extension reaction; and
(e) optionally circularizing and transforming a host cell with the fully complementary, chimeric DNA duplex of step (d).
